(19) United States
(12) Patent Application Publication
(10) Pub. No.: US 2010/0167282 A1
(43) Pub. Date: Jul. 1, 2010

(54) LATERAL-FLOW DEVICE AND METHOD TO ENHANCE DETECTION SIGNAL FOR CHARGED MOLECULAR TARGETS

(76) Inventors: Jui-Chuang Wu, Tao-Yuan (TW); Huan-Ching Yang, Taipei (TW)

Correspondence Address: STOUT, UXA, BUYAN & MULLINS LLP, 4 VENTURE, SUITE 300, IRVINE, CA, 92618, US

(21) Appl. No.: 12/396,429

(22) Filed: Mar. 2, 2009

(30) Foreign Application Priority Data
Dec. 31, 2008 (TW) 097151595

(51) Int. Cl. C12Q 1/68; C12M 1/34

(52) U.S. Cl. 435/6; 435/287.2

(57) ABSTRACT

The present invention proposes a lateral-flow device and method to enhance the detection signal for charged molecular targets. The device includes a detection assembly and a direct current source. The direct current is applied on the device to reduce the amount of residual target on the membrane and the detection limit is thus improved.

### BACKGROUND OF THE INVENTION

1. Field of the Invention

The present invention generally relates to a detection device and method for charged molecular targets, and more particularly to a membrane based lateral-flow device and method to enhance the detection signal for charged molecular targets, to enhance the detection sensitivity.

2. Description of the Prior Art

Immunochromatography is an easy and fast detection method, and users, even when not well-trained, can determine test results without the need for or use of any extra instruments. Because of its convenient operation and relatively quick readout capability, immunochromatography is wildly applied in the fields of environment testing, food testing, bio-chemical weapons, clinical medicine, etc. According to the capillarity of different membranes with different density and specific immunity between targets and marked reagents, the color of the marked reagents can be read to indicate whether the targets exist, and even to show the concentration of the targets.

The above-mentioned immunochromatography characteristics are based upon antigen-antibody immunoassay considerations. Similarly, the binding specificity between DNA-ligand and anti-ligand bodies is employed to detect nucleic acid targets by the hybridization reaction in a rapid and convenient manner.

FIG. 1 shows a conventional rapid DNA detection device. First, the user drips down the dig-labeled single-stranded DNA molecular targets 51 to the sample pad 10. The molecular targets 51 flow to the conjugate pad 20 because of the capillarity-induced movement, and the dig-labeled molecular targets 51 combine with the first antibody 53 which possesses the label. Then, the combining complex continues to flow to the membrane pad 30, and the complementary DNA probe immobilized in the test line 32 will capture the single-stranded DNA of the combining complex. Thus the color of the label in the test line 32 can be distinguished visually or detected by instruments. The first antibodies 53, those not conjugated with the molecular targets 51, will flow forward to the control line 34, and will be captured by the secondary antibody immobilized in the control line 34. Thus the color of the label can be distinguished visually or detected by instruments. Because of capillarity, the rest of the first antibody 53 as well as the fluid placed in the sample pad will continue to flow to the absorbent pad.

Nitrocellulose is usually used for the material of the lateral-flow membrane. Since the nitrocellulose has strong affinity to the molecular targets, the capillarity-induced movements of some molecular targets are trapped in the membrane. Therefore, not all targets flow to the test line and show its distinguished color, and the threshold of the detection limit is not ideal. Only molecular targets with high concentration in samples suit this traditional detection approach. In this regard, it is necessary to provide an improvement method for reducing the remains of the molecular targets on the membrane to increase the sensitivity.

### SUMMARY OF THE INVENTION

In view of the foregoing omission of the prior art, one object of the present invention is to provide a membrane based lateral-flow detection device for charged molecular targets with a sensitive detection limit. This detection device includes a membrane based lateral-flow detection assembly and a direct current source.

Another object of the present invention is to enhance the movement of the charged molecular targets by the direct current source, whereby the detection sensitivity is therefore increased.

According to the foregoing objects, the present invention provides a membrane based lateral-flow method and device. This membrane based lateral-flow device includes a membrane based lateral-flow detection assembly and a direct current source. With the adding of direct current, the movement of the charged molecular targets is enhanced, the remains of the molecular targets on the membrane pad are reduced, and the detection limit is increased.

### BRIEF DESCRIPTION OF THE DRAWINGS

FIG. 1 is a cross-sectional view showing a conventional rapid detection assembly for DNA.

FIG. 2 is a schematic diagram illustrating a membrane based lateral-flow device for charged molecular targets of the present invention.

FIG. 3A is a graphic view illustrating the result of a conventional membrane based lateral-flow detection device, without any voltage added.

FIG. 3B is a graphic view showing the result of a membrane based lateral-flow device for charged molecular targets of the present invention.

FIG. 4 is a graph comparing the result with that on a conventional gel-electrophoresis.

FIG. 5 is a flow chart illustrating steps of a membrane based lateral-flow device for charged molecular targets of the present invention.

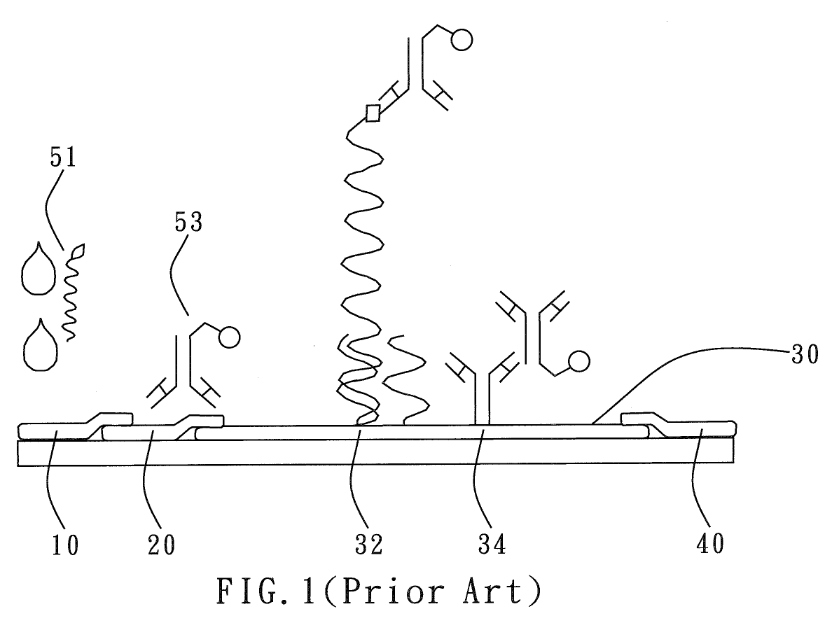

### DETAILED DESCRIPTION OF THE INVENTION

A detailed description of the present invention will be provided in connection with the following embodiments, which are not intended to limit the scope of the present invention and which can be adapted to other applications. While the drawings are illustrated in detail, it is appreciated that the quantity of the disclosed components may be greater or fewer than that disclosed, except where the amount of such components is expressly restricted.

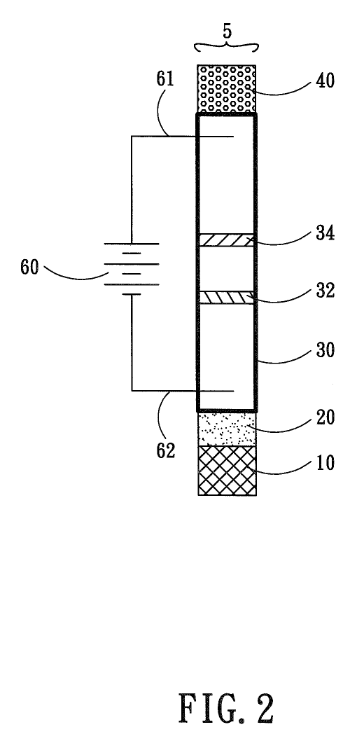

In accordance with an embodiment of the present invention, FIG. 2 shows a membrane based lateral-flow device for charged molecular targets. The membrane based lateral-flow device includes a membrane based lateral-flow detection assembly 5 and a direct current source 60. The membrane based lateral-flow detection assembly 5 includes a sample pad 10, a conjugate pad 20, a membrane pad 30, a test line 32, a control line 34, and an absorbent pad 40. As shown, from top to bottom, the membrane based lateral-flow detection assembly 5 includes the sample pad 10, the conjugate pad 20, and the absorbent pad 40 in sequence, and the test line 32 and the control line 34 are on the membrane pad 30. In addition, the direct current source 60 includes the first electrode 62 and the second electrode 61, and the electricity of the first electrode 62 is different from that of the second electrode 61. The first electrode 62 is located between the conjugate pad 20 and the test line 32, and the second electrode 61 is located between the control line 34 and the absorbent pad 40. The direct current source provides an electromotive force (EMF) to drive the charged molecular targets flowing from the first electrode 62 to the second electrode 61, and the electricity of the charged molecular targets is different from that of the second electrode 61.

When the electricity of the charged molecular targets is positive, the first electrode should be chosen to be an anode, and the second electrode should be chosen to be a cathode. On the contrary, when the electricity of the charged molecular targets is negative, the first electrode should be chosen to be a cathode, and the second electrode should be chosen to be an anode. The direct current source can come from the batteries, or provided by the inverter/transformer converting from the alternating current.

With comparative reference to FIG. 2, in another embodiment the user drips down the dig-labeled single-stranded DNA molecular targets in the sample pad 10. The molecular targets flow to the conjugate pad 20 because of capillarity-induced movement, and the dig-labeled molecular targets combine with the first antibody which possesses the label. Then, the combining complex flows to the membrane pad 30, and the complementary DNA probe immobilized in the test line 32 will capture the single-stranded DNA of the combining complex; thus the color of the label can be distinguished visually. In this embodiment, the second electrode 61 (anode) is located between the control line 34 and the absorbent pad 40, and the first electrode 62 (cathode) is located between the conjugate pad 20 and the test line 32. Because of the DNA being with negative charge, the electromotive force caused by the direct current source 60 will drive DNA to flow to the absorbent pad 40, and the remains of DNA on the membrane pad 30 will be reduced. Furthermore, the color on the test line 32 will be more true, apparent or obvious, and the detection sensitivity is therefore enhanced.

The foregoing conjugate pad includes a label, a labeled antibody, and a labeled secondary antibody or a labeled antigen, wherein the label includes fixed-radioactive substance (such as I¹²⁵, H³, C¹⁴ and P³²), enzyme, fluorescence material, dye, carbon black, colloid gold and latex.

The foregoing molecular targets are not limited by the embodiment of the present invention. Those charged samples suit the detection device revealed by the present invention, such as Deoxyribonucleic acid (DNA), RiboNucleic Acid (RNA), protein, amino acid, bio-molecules, medicine, drugs and specialty chemicals.

### EXAMPLE

The avian influenza is chosen to be the target, and the DNA targets are H5 sequence in A/Singapore/1/57 (H2N2). First, single-stranded DNA product is amplified by asymmetrical Polymerase Chain Reaction (PCR) and dig-labeled. After purification, targets react with the anti-ligand antibody which is labeled with a color label (e.g., colloid gold). Then, targets flow to the test zone, and hybridization reaction is performed to show the color of colloid gold. The detection result is designed to be visually read out by gold-sol to identify distinct sorts of Avian Influenza.

Viruses such as avian influenza have RNA genomes that can be converted into complementary DNA (cDNA) by an enzyme called reverse transcriptase. cDNA is a more convenient way to work with the coding sequence than mRNA because RNA is very easily degraded by omnipresent RNases. This is the main reason why cDNA is sequenced rather than mRNA. Likewise, investigators conducting DNA microarrays often convert the mRNA into cDNA in order to produce their probes. The cDNA of avian influenza is then amplified by asymmetrical PCR, and analyzed by agarose gel electrophoresis.

Since the segment of DNA is 358 mer in length and might move slow (e.g., slowly) down (e.g., downwardly) due to its bulky structure, a direct current voltage is added to enhance the detection signal.

A complementary probe cH5 is included in the test line 32 of the present invention. The control line possesses Rabbit anti-mouse IgG. The conjugate pad 20 possesses mouse anti digoxigenin labeled nano-gold which reacts with the dig part in the targets cH5. The sample pad 10 contains dig-labeled H5 PCR product.

The probe cH5 in the test line 32 will hybridize with the PCR product, and digoxigenin of PCR product will capture gold-conjugated mouse anti digoxigenin to show the color of nano-gold. Besides, Rabbit anti-mouse IgG in the control line 34 will react with gold conjugated mouse anti digoxigenin to show the color of nano-gold.

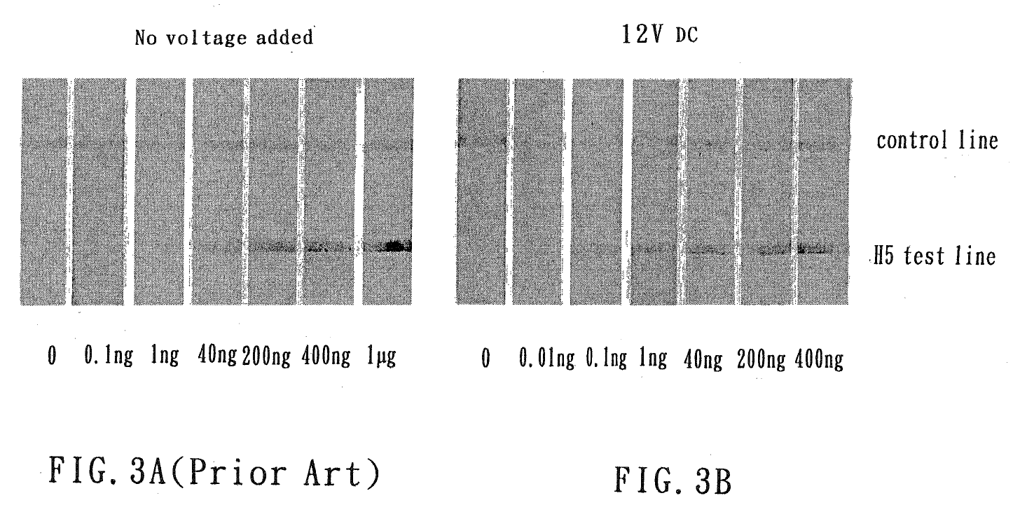

FIG. 3A and FIG. 3B illustrate the comparison results of membrane based lateral-flow detection devices of a conventional system and the present invention. In FIG. 3A, without any voltage added, it is distinguished on the test line where the concentration of H5 can be virtualized about (or greater than) 40 ng/100 μl. In FIG. 3B, with 12V DC added, it is distinguished on the test line where the concentration of H5 can be virtualized about (or greater than) 0.1 ng/100 μl. Apparently, the provided membrane based lateral-flow detection device of the present invention reduces the remains of the charged molecular targets and enhances the detection sensitivity.

Generally, the result of agarose gel-electrophoresis is very important to bio-molecule detection limit. Since the principle of the membrane based lateral-flow detection device for the charged molecular targets in the present invention is something similar to the agarose gel-electrophoresis, it is necessary to execute an agarose gel-electrophoresis experiment with different H5 concentration for comparison. 2% Agarose gel and 30 ml 0.5× TBE buffer solution are used for 35 minutes gel-electrophoresis, then Agarose gel is soaked in Ethidium bromide (EtBr) for 20 minutes. Finally, UV lamp or UV lightbox is used to visualize DNA in the gel.

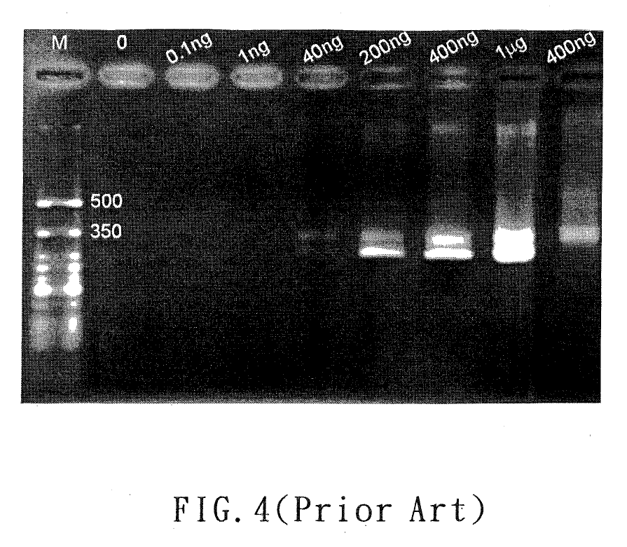

As shown in FIG. 4, in the gel-electrophoresis, the detection threshold is 40 ng/100 μl. That is, the gel-electrophoresis detection only suits the concentration of H5 that is (or is greater than) 40 ng/100 μl. It is the same as the membrane based lateral-flow detection device without direct current source added. Clearly, the detection sensitivity of the present invention is superior to the conventional gel-electrophoresis.

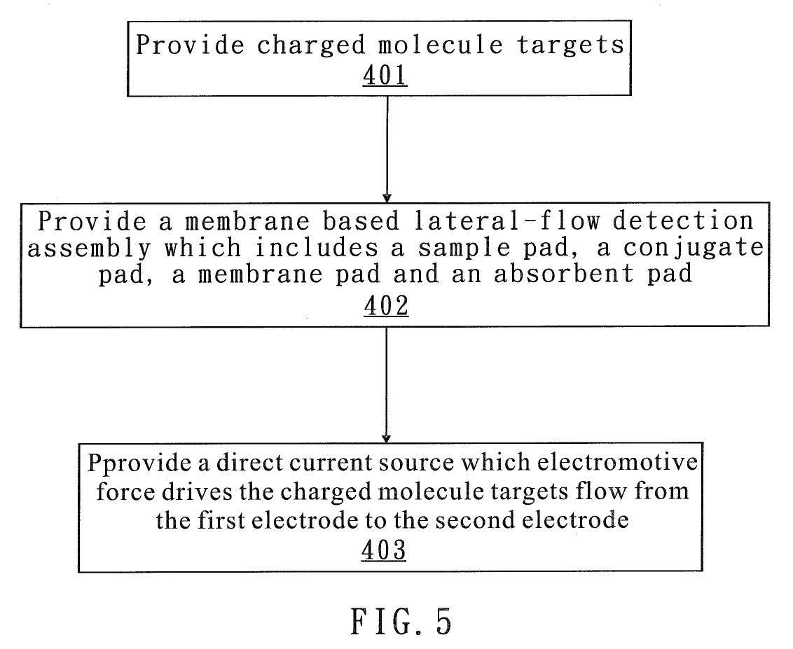

FIG. 5 is a flowchart showing steps of a membrane based lateral-flow device for charged molecular targets according to the present invention. In step 401, the charged molecular targets are provided, and if the molecular targets are some kind of protein or amino acid the electricity can be changed by adjusting the pH of the molecular targets to something away from their iso-electric point such as: acidifying or alkalizing the molecular targets. In step 402, a membrane based lateral-flow detection assembly which includes a sample pad, a conjugate pad, a membrane pad, and an absorbent pad is provided, wherein the membrane pad possesses a test line and a control line.

In step 403, a direct current source which includes a first electrode and a second electrode is provided, wherein the electricity of the first electrode is different from that of the second electrode. The first electrode is located between the conjugate pad and the test line, and the second electrode is located between the control line and the absorbent pad. The direct current source provides an electromotive force to drive the charged molecular targets to flow from the first electrode to the second electrode, and the electricity of the charged molecular targets is different from that of the second electrode.

Although specific embodiments have been illustrated and described, it will be appreciated by those skilled in the art that various modifications may be made without departing from the scope of the present invention, which is intended to be limited solely by the appended claims.

What is claimed is:

1. A membrane based lateral-flow detection device for charged molecular targets, comprising:
a membrane based lateral-flow detection assembly, said membrane based lateral-flow detection assembly comprising a sample pad, a conjugate pad, a membrane pad and an absorbent pad, and said membrane pad comprising a test line and a control line; and
a direct current source, said direct current source comprising a first electrode and a second electrode, wherein the electricity of said first electrode is different from the electricity of said second electrode, said first electrode is located between said conjugate pad and said test line, and said second electrode is located between said control line and said absorbent pad, and wherein said charged molecular targets, with electricity different from the electricity of said second electrode, are forced to flow from said first electrode to said second electrode.

2. The membrane based lateral-flow detection device for charged molecular targets according to claim 1, wherein said direct current source provides an electromotive force to drive the charged molecular targets to flow from said first electrode to said second electrode.

3. The membrane based lateral-flow detection device for charged molecular targets according to claim 1, wherein the electricity of said molecular targets is positive, said first electrode is an anode, and said second electrode is a cathode.

4. The membrane based lateral-flow detection device for charged molecular targets according to claim 1, wherein the electricity of said molecular targets is negative, said first electrode is a cathode, and said second electrode is an anode.

5. The membrane based lateral-flow detection device for charged molecular targets according to claim 1, wherein said conjugate pad comprises a label, a labeled antibody, and a labeled secondary antibody or a labeled antigen, and the label is selected from the group consisting of fixed-radioactive substance, enzyme, fluorescence material, dye, carbon black, colloid gold and latex.

6. The membrane based lateral-flow detection device for charged molecular targets according to claim 1, wherein said molecular target is selected from the group consisting of Deoxyribonucleic acid (DNA), RiboNucleic Acid (RNA), protein, amino acid, bio-molecules, medicine, drugs and specialty chemicals.

7. The membrane based lateral-flow detection device for charged molecular targets according to claim 1, wherein said membrane based lateral-flow detection assembly comprises said sample pad, said conjugate pad, said membrane pad and said absorbent pad in sequence.

8. The membrane based lateral-flow detection device for charged molecular targets according to claim 1, wherein said direct current source is at least one battery or said direct current source is generated by an inverter/transformer converting from an alternating current.

9. The membrane based lateral-flow detection device for charged molecular targets according to claim 1, wherein the material of the membrane pad is selected from the group consisting of cellulose, cellulose acetate, PVDF, polypropylene, polyurethane, polyacrylonitrile, nitrocellulose and polysulfone.

10. A membrane based lateral-flow detection method to enhance the detection signal for charged molecular targets, comprising:
providing a plurality of charged molecular targets;
providing a membrane based lateral-flow detection assembly, said membrane based lateral-flow detection assembly comprising a sample pad, a conjugate pad, a membrane pad and an absorbent pad, and said membrane pad comprising a test line and a control line; and
providing a direct current source, said direct current source comprising a first electrode and a second electrode, wherein the electricity of said first electrode is different from the electricity of said second electrode, said first electrode is located between said conjugate pad and said test line, and said second electrode is located between said control line and said absorbent pad, and wherein said charged molecular targets, with electricity different from the electricity of said second electrode, are forced to flow from said first electrode to said second electrode.

11. The membrane based lateral-flow detection method to enhance the detection signal for charged molecular targets according to claim 10, further comprising electrifying the molecular targets by adjusting the pH of said molecular targets in the step of providing said charged molecular targets.

12. The membrane based lateral-flow detection method to enhance the detection signal for charged molecular targets according to claim 10, wherein said direct current source provides an electromotive force to drive the charged molecular targets to flow from said first electrode to said second electrode.

13. The membrane based lateral-flow detection method to enhance the detection signal for charged molecular targets according to claim 10, wherein the electricity of said molecular targets is positive, said first electrode is an anode, and said second electrode is a cathode.

14. The membrane based lateral-flow detection method to enhance the detection signal for charged molecular targets according to claim 10, wherein the electricity of said molecular targets is negative, said first electrode is a cathode, and said second electrode is an anode.

15. The membrane based lateral-flow detection method to enhance the detection signal for charged molecular targets according to claim 10, wherein said conjugate pad comprises a label, a labeled antibody, and a labeled secondary antibody or a labeled antigen, and the label is selected from the group consisting of fixed-radioactive substance, enzyme, fluorescence material, dye, carbon black, colloid gold and latex.

16. The membrane based lateral-flow detection method to enhance the detection signal for charged molecular targets according to claim 10, wherein said molecular targets are selected from the group consisting of Deoxyribonucleic acid (DNA), RiboNucleic Acid (RNA), protein, amino acid, bio-molecules, medicine, drugs and specialty chemicals.

17. The membrane based lateral-flow detection method to enhance the detection signal for charged molecular targets according to claim 10, wherein said membrane based lateral-flow detection assembly comprises said sample pad, said conjugate pad, said membrane pad and said absorbent pad in sequence.

18. The membrane based lateral-flow detection method to enhance the detection signal for charged molecular targets according to claim 10, wherein said direct current source is at least one battery or said direct current source is generated by an inverter/transformer converting from an alternating current.

19. A membrane based lateral-flow detection device for charged molecular targets, comprising:
a membrane based lateral-flow detection assembly, said membrane based lateral-flow detection assembly comprising a sample pad, a conjugate pad, a membrane pad and an absorbent pad, said membrane pad comprising a test line and a control line; and
a direct current source connected to said membrane pad, said direct current source comprising a cathode and an anode, wherein said cathode is located between said conjugate pad and said test line, said anode is located between said control line and said absorbent pad, and said charged molecular targets are forced to flow from said cathode to said anode by an electromotive force provided by said direct current source.

20. The membrane based lateral-flow detection device for charged molecular targets according to claim 19, wherein said molecular targets are selected from the group consisting of Deoxyribonucleic acid (DNA), RiboNucleic Acid (RNA), protein, amino acid, bio-molecules, medicine, drugs and specialty chemicals.

21. The membrane based lateral-flow detection device for charged molecular targets according to claim 19, wherein said membrane based lateral-flow detection assembly comprises said sample pad, said conjugate pad, said membrane pad and said absorbent pad in sequence.

22. The membrane based lateral-flow detection device for charged molecular targets according to claim 19, wherein the material of the membrane pad is selected from the group consisting of cellulose, cellulose acetate, PVDF, polypropylene, polyurethane, polyacrylonitrile, nitrocellulose and polysulfone.
